(19) United States
(12) Patent Application Publication
(10) Pub. No.: US 2010/0080853 A1
(43) Pub. Date: Apr. 1, 2010

(54) Use of Spongilla Spicules as a Dermabrasion Device or Resurfacing Modality

(76) Inventor: Maria Villani, Newsport Coast, CA (US)

Correspondence Address: K&L Gates LLP, 1900 MAIN STREET, SUITE 600, IRVINE, CA, 92614-7319, US

(21) Appl. No.: 12/444,495

(22) PCT Filed: Oct. 5, 2007
(86) PCT No.: PCT/US07/80587

§ 371 Date: Apr. 6, 2009

Related U.S. Application Data
(60) Provisional application No. 60/828,324, filed on Oct. 5, 2006.

(51) Int. Cl. A61K 35/00; A61P 17/00

(52) U.S. Cl. 424/520

(57) ABSTRACT

Dermabrasive compositions for skin resurfacing are disclosed. Therapeutic compositions disclosed are derived from Porifera species, specifically sponges, and more specifically fresh water sponges. One disclosed embodiment is derived from Spongilla species and is formulated with pharmaceutically acceptable excipients.

### CROSS REFERENCE TO RELATED APPLICATIONS

This application claims the benefit under 35 U.S.C. §119(e) to U.S. Provisional Patent Application No. 60/828,324 filed Oct. 5, 2006 which is incorporated by reference herein in its entirety.

### FIELD OF THE INVENTION

The present invention is directed to dermabrasive compositions suitable for treating skin diseases. Specifically, the dermabrasive compositions of the present invention are purified from fresh water sponges of the phylum Porifera. The dermabrasive compositions of the present invention are applied topically for the treatment of various skin disorders and diseases.

### BACKGROUND OF THE INVENTION

One of the manifestation of aging skin is decreased ability to shed dead cells, resulting in various unsightly skin conditions. The mainstay of topical therapy of photo-aging skin is the chemical peel. A chemical peel is a procedure in which a topically applied wounding agent creates smooth, rejuvenated skin by way of an organized repair process. Complications of chemical resurfacing, including permanent sequelae, such as pigmentary dyschromias, infection, or scarring, may occur even though a controlled chemical wound induced.

Dermabrasion helps to “refinish” the skin's top layers through a method of controlled surgical scraping. The treatments soften the sharp edges of surface irregularities, giving the skin a smoother appearance. Dermabrasion is most often used to improve the look of facial skin left scarred by accidents or previous surgery, or to smooth out fine facial wrinkles, such as those around the mouth. It's also sometimes used to remove the pre-cancerous growths called keratoses. Dermabrasion can be performed on small areas of skin or on the entire face. It can be used alone, or in conjunction with other procedures such as facelift, scar removal or revision, or chemical peels. Typically dermabrasion is conducted under anesthetic.

Microdermabrasion is a cosmetic procedure in which the stratum corneum is partially or completely removed by light abrasion. Different methods include mechanical abrasion from jets of zinc oxide or aluminum oxide crystals, fine organic particles, or a roughened surface. Microdermabrasion is used to remove sun-damaged skin and to remove or lessen scars and dark spots on the skin. The procedure is not very painful and requires no anaesthetic. Microdermabrasion can be used medically for scar removal when the scar is raised above the surrounding skin, but is less effective with sunken scars.

### SUMMARY OF THE INVENTION

The present invention relates to dermabrasive skin care compositions purified from invertebrate species of the phylum Porifera. In one embodiment of the present invention, a dermabrasive composition for skin is provided comprising a substantially pure powder of a fresh water sponge of the genus Spongilla and at least one pharmaceutically acceptable excipient, wherein the composition comprises insoluble material of the substantially pure powder of Spongilla and wherein the insoluble material comprises skeletal spicules.

In one embodiment, the freshwater sponge is of the species Spongilla lacustris. In another embodiment, the pharmaceutically acceptable excipient is a carrier gel or liquid. In another embodiment, the substantially pure powder does not contain any particles larger than 0.2 mm. In yet another embodiment, the dermabrasive composition comprises from 0.1% to 100% substantially pure Spongilla powder.

In another embodiment of the present invention, the pharmaceutically acceptable excipient is selected from the group consisting of water, glycerin, gels, oils, waxes, emollients, cleansers, fragrances, antiseptics, anesthetics, seaweed powder, coral powder, hydrogen peroxide, enzyme gel, jojoba oil and boric acid. In another embodiment, water is selected from the group consisting of water for injection, irrigation water, distilled water, deionized water, chamomile water and calendula water.

### BRIEF DESCRIPTION OF THE DRAWINGS

FIG. 1 graphically depicts different spicule structures occurring in marine and fresh water sponges of the present invention.

FIG. 2A-B are scanning electron micrographs of spicules occurring in marine and fresh water sponges of the present invention.

FIG. 3 depicts the physical appearance of the skin of a subject before (FIG. 3A) and after (FIG. 3B) skin resurfacing with the dermabrasive composition of the present invention.

### DETAILED DESCRIPTION OF THE INVENTION

Spongilla spicules are effective modality of skin resurfacing and provide a natural alternative approach to microdermabrasion and dermabrasion, chemical peels (glycolic acid, Jessner's, phenol and trichloroacetic acid peels), CO₂ laser resurfacing and Erbium laser peels to correct visual facial defects such as hyperpigmentation of various etiology, fine wrinkles, sun damage, and superficial scars, comedones, enlarged pores and facial rejuvenation. Upon massaging Spongilla spicules into the skin, the spicules mechanically separate the epidermal layers and reduce keratinocyte cohesion, thereby increasing stratum corneum sloughing and sebum plug and loose keratinocyte removal.

The present invention relates to novel skin care therapeutics derived from invertebrate species of the phylum Porifera. Poriferans are commonly referred to as sponges. The approximately 5,000 living sponge species are classified in the phylum Porifera, which is composed of three distinct groups, the Hexactinellida (glass sponges), the Demospongia, and the Calcarea (calcareous sponges).

Sponges are multicellular marine animals belonging to a large group of simple animal species known as invertebrates. Sponges originated billions of years ago and are among the oldest animals on earth. Presently, approximately 5,000 species of Sponges are known. Sponges are composed of a soft tissue suspended in a jelly-like proteinaceous matrix supported by a hard skeleton composed of needle-like structures known as spicules. Spicules are primarily composed of calcium carbonate, or silica and collagen.

Fresh water sponges suitable for use in the compositions of the present invention include, but not limited to, sponges belonging to the species Spongilla lacustris L., Spongilla fragilis Leidy, and Ephydatia fluviatilis.

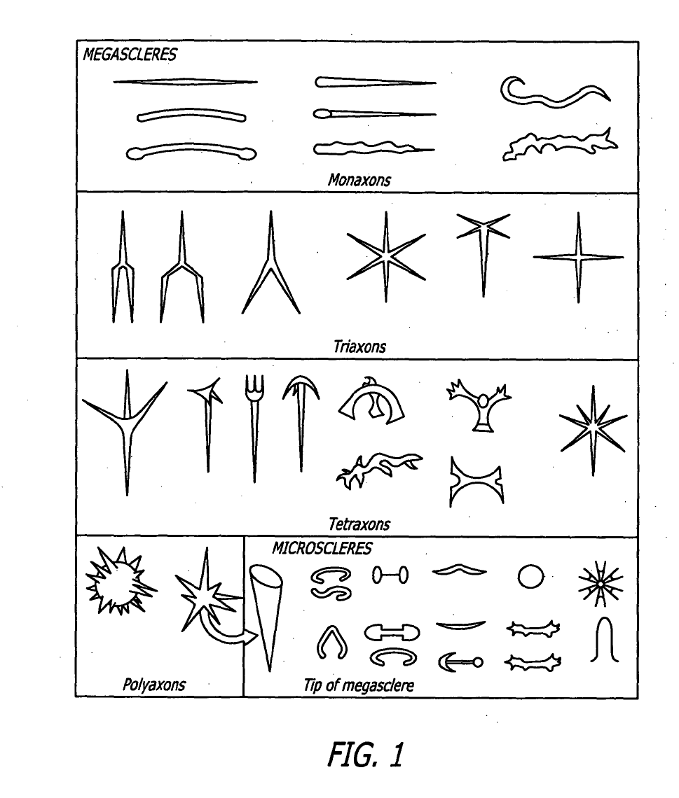

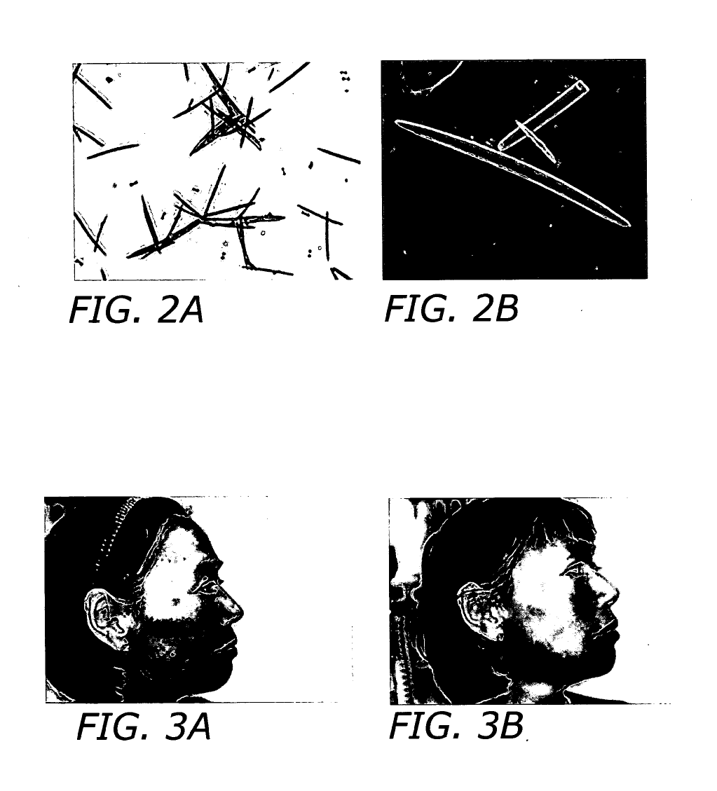

Spicules are skeletal structures that occur in most marine and fresh water sponges (FIG. 2). They provide structural support for the sponges. Large spicules, visible to the naked eye are referred to as megascleres, while smaller, microscopic ones are termed microscleres (FIG. 1). Spicules have four major symmetry types: Monaxon (simple cylinders with pointed ends), triaxon, tetraxon, and polyaxon. Sponges can be calcareous, siliceous or composed of spongin. The meshing of numerous spicules serves as the sponge's skeleton. The composition, size, and shape of spicules are the largest determining factors in sponge taxonomy.

Compositions made in accordance with the teachings of the present invention have been analyzed extensively. The dried substantially purified powder of the present invention is an odorless, grayish-red non-hygroscopic powder. The powder is partially soluble in water and forms a greenish-red colored solution when mixed in a ratio of 1 part to 3; approximately 50 to 60% percent remains insoluble and comprises the organic fraction including spicules with mechanical-abrasive properties. The pH of the soluble fraction is between approximately 7.0 to 7.5 with a mean pH of 7.35; the specific gravity is between approximately 1.04 to 1.07 with a mean specific gravity of 1.058. Peak absorption is observed at between 210 nm to 250 nm when measured between 200 and 900 nm using methods known to those skilled in the art of physical chemistry.

Table 1 includes a non-limiting representative analysis of the organic and inorganic constituents.

TABLE 1*

| Inorganic | IC mg/g of | Organic | OC g/110 g of | | Units |
|---|---|---|---|---|---|
| Component | dried raw | Component | dried raw | Enzyme Activity | per |
| (IC) | material | (OC) | material | (EA) | 100 g |
| Sodium | 160-170 | Protein | 1.90-2.00 | Alkaline | 80-90 |
| | | | | Phosphatase | |
| Potassium | 120-130 | Neutral fats | 1.10-1.20 | Asparagine | 20-25 |
| | | | | Transferase | |
| Ammonia | 30-40 | Glucose | 0.3-0.4 | Alanine | 9-10 |
| | | | | Transaminase | |
| Calcium | 160-170 | Steroids | 0.0002 | Gamma-glutamyl | 7-8 |
| | | | | Transpeptidase | |
| Magnesium | 20-40 | Hydroxy- | Trace | Catalase | 50-55 |
| | | purines | | | |
| Iron | 320-330 | Total Nitrogen | 0.012-0.014 | Malanic dialdehyde | 0.15-0.2 |
| Copper | 190-200 | | | Superoxide | 6030-6040 |
| | | | | dismutase | |
| Zinc | 11-13 | | | Ceruloplasmin | 450-500 |
| Chlorine | 130-140 | | | | |
| Sulfate | 115-120 | | | | |
| Phosphate | 420-430 | | | | |
| Nitrate | 25-30 | | | | |
| Bicarbonate | 540-550 | | | | |
| Carbonate | 120-125 | | | | |
| Silicates | 13-15 | | | | |

*Ranges are approximate and based on normal laboratory standard deviations for the assay methods employed. Assays were conducted using standard analytical procedures known to those having ordinary skill in the art of analytical chemistry. Enzyme activity is expressed in units of enzyme activity per 100 mg of dried material. Units are based on a recognized International Biological Standard for each assay.

It should be understood by those skilled in the art that the elemental and organic analysis performed on representative samples is not intended to be a comprehensive or even partial listing of the active ingredients found in the compositions of the present invention. As previously discussed, there may be myriad bioactive molecules present in the Porifera products of the present invention that have not been previously identified. The analytical data in Table 1 provides persons skilled in the art non-limiting data that may be useful in characterizing compositions made in accordance with the teachings of the present invention. However, in addition to other possible synergetic and complementary bioactive compounds contained in the present invention the ingredients identified in Table 1 may also provide certain beneficial effects. Without being bound to this theory, the present inventor proposes a possible role for many of the quantified ingredients in Table 1.

In an exemplary embodiment of the present invention the Porifera is used to prepare topical dermabrasives is Spongilla lacustris. As used herein, “substantially pure” refers to a Porifera sp. that has been purified of environmental debris including rocks, sticks, other marine life etc., washed, dried, ground, sieved and sized to form a uniform, reproducible manufactured material.

Spongilla lacustris is generally preferred for making the compositions of the present invention because this sponge genus is highly tolerate of natural environmental variation and grows extremely well in a wide range of habitats.

Once an appropriate aquatic environment and sponge habitat is identified sponge collection can begin using methods commonly known to those skilled in the art of marine biology. For example, sponges can be collected manually using basic under water diving techniques, or in deeper waters larger colonies are harvested using the Agassiz trawl (AGT) or epibenthic sledge (EBS). However, sponges smaller than 0.5 cm in diameter are unlikely to be collected by AGT. Under certain environmental conditions S. lacustris colonies occur in a thin crust-like carpet several meters across and must be collected manually.

Freshly collected sponges removed from their aquatic habitat are mucoid amorphous masses and emit a characteristic odor that most observers describe as unpleasant. Before the collected sponge mass is dried it must be clean of gross contamination including portions of the substrata, shells, stems, plants, small fresh water animals, rocks and other impurities. Next the sponge mass is washed to remove dirt, sand, silt and soluble impurities. The wash water is changed repeatedly until it is clear and the sponges appear free from contamination. After removing gross debris and cleaning, the sponge mass is weighed and dried. As an alternative, collected sponge colonies can be sent to the repository, a low-temperature storage facility for their quarantine, delayed processing and further investigation.

When dried under ambient, open-air conditions temperature, dew point, relative humidity and forecasted precipitation must be closely monitored. If the ambient air temperature is too low or if precipitation is forecasted the sponge mass should be dried inside where temperature and humidity can be controlled. It is not essential that a precise temperature or humidity range be maintained, however, the sponge mass should be maintained within a temperature and humidity range suitable for an uninterrupted evaporative process to proceed. For example, temperatures should be above 60° F. and relative humidity should be below 90%. However, it is recommended that the sponge mass should be protected from exposure to atmospheric precipitation and excessive temperatures after collection. The sponge mass is dried until residual moisture content is less than 10%, preferably less than 5%. If the raw material is to be stored for protracted periods before further processing, residual moisture can be as low a 0.1% or less. Residual moisture measurements can be performed using methods commonly known in the arts of food sciences, analytical chemistry or the pharmaceutical sciences. For example, 10 grams of dried material is placed on a tared weighing boat and then weighed. The weighed material is then exposed to a heat source such as a drying oven or heat lamp operated at a temperature sufficient to evaporate any remaining free or loosely bound water (non-chemically bound). The sample is then cooled in a desiccated chamber and re-weighed. Residual moisture is calculated as the percent difference between the sample weight before drying and the weight after cooling.

Once dried, the sponge is packaged in sealed containers, protected from light and maintained in quarantine at 55° to 75° F. Routine quality control processes are conduced on the dried sponge material consistent with Good Manufacturing Practice Requirements (GMP) and International Standards Organization (ISO) regiments applicable to food, drugs and cosmetics before being released from quarantine and processed further. Testing includes microbiological culturing for pathogens, coliform organisms and bioburden. Chemical analysis is also performed to verify the product's identity, potency and purity. All processing after the initial drying phase should be conducted in environmentally controlled facilities that comply with GMP and ISO guidelines. Manufacturing personnel must be trained in GMP and ISO procedures and all manufacturing process closely monitored and recorded.

After release from quarantine the raw, dried sponge material is further purified, refined and processed to a standard particle size using sieves. The dried sponge is extremely fragile and requires only slight, gentle grinding to form a consistently fine particulate. The dried sponges collected and processed in accordance with the teachings of the present invention should not be processed using aggressive grinding techniques, rather the dried sponge is processed gently to avoid crushing debris that may be present in the sample. For example, shells from aquatic mollusks may contaminate the crude sample; grinding of the crude sponge preparation should be conducted in a fashion that will not pulverize the contaminating shells to a degree that they would not be removed in the sieving processes. Several grinding and sieving steeps are performed to reduce average particle size to no more than 0.2 mm. First a course grind and sieving process is used to reduce particle size to at least 2 mm. This initial sieving process also permits visual inspection and removal of remaining non-sponge debris and is followed by subsequent grinding and sieving processes where the raw material is ultimately reduced to no more than 0.2 mm particles.

Next the sized material is ground and sieved again to reduce particle size to no more than 0.2 mm. After the desiccated sponge powder is ground, it is further purified and separated from contaminants by processing the powder with sieves having progressively smaller apertures (1 mm, 0.5 mm, and 0.2 mm respectively). All processing is conducted under GMP conditions.

After final grinding and sizing processes are completed the purified dried sponge material is packaged in airtight moisture-proof containers and stored in the dark at 55° to 75° F. under desiccated conditions. No preservatives are required due to the natural antimicrobial properties of the Spongilla powder. The purified Spongilla powder collected, processed and stored in accordance with the teachings of the present invention is stable for a minimum of three years and six months. However, accelerated testing data may suggest much longer stability periods (up to 10 years).

The dermabrasive compositions of the present invention comprise from approximately 0.1% to 100% substantially pure Spongilla powder and can optionally compounded with pharmaceutical excipients including, but not limited to water, saline, buffered phosphate, oils, gels, waxes, emollients, glycerin, cleansers, fragrances, colorings, antiseptics and anesthetics. Suitable waters include water for injection, irrigation water, distilled water, deionized water, and floral water among others. Even clean tap water is acceptable for some applications. The concentrations of the aforementioned excipients can range from 0.001% to 50% or more depending on the requirements and at the discursion of the formulation scientist, pharmacist or prescribing physician. Such ranges are well known in the art and can be determined without undue experimentation. Other excipients that may be used in accordance with the teachings of the present invention may include from approximately 0.1% to 25% coral powder, from approximately 0.1% to 25% seaweed powder, from approximately 0.1% to 10% hydrogen peroxide and from approximately 0.1 to 10% of an inorganic or organic acid such as, but not limited to boric acid, hydrochloric, ascorbic acid, salicylic acid, and others.

The therapeutic compositions of the present invention generally comprise from 0.8 to 1.5 grams of substantially pure Spongilla powder, and at least one additional excipient selected from the group consisting of from 0.1 to 0.5 grams of green seaweed powder, from 0.1 to 0.5 grams of white seaweed powder, from 0.1 to 0.5 grams of coral powder, from 0.1 to 0.5 grams of Plantain powder, from 0.5 mL to 5 mL of 0.1% to 10% hydrogen peroxide, from 0.5 mL to 5 mL of 0.1% to 10% boric acid and from 0.5 to 5 mL of water, from 0.5 mL to 5 mL enzyme gel (comprising water, hydroxyethylcellulose, hyaluronic acid, propylene glycol, methylparaben, tetrasodium EDTA and propylparaben in proportions suitable for topical applications as known to those skilled in the art), from 0.5 mL to 10 mL jojoba oil. Other excipients such as, but not limited to saline, buffered phosphate, oils, waxes, emollients, glycerin, cleansers, fragrances, colorings, antiseptics and anesthetics may be added as desired or required.

Upon massaging substantially pure Spongilla powder into the skin, spicules penetrate epidermis creating controlled microscopical injuries to the skin. By doing so, they activate a wound healing responses which are normally take place in a natural restorative response to tissue injury. Observed erythema is due to the increased blood circulation in the treated skin. Vasodilation is an important means by which the wound can be exposed to increased blood flow, accompanied by the necessary inflammatory cells and factors that fight infection and debride the wound of devitalized tissue. Increased blood circulation helps to dissolve bruises, stagnant spots and infiltrates

Wound healing responses stimulate new cell growth, elastin and collagen production and improve skin tone and texture.

Penetrating spicules additionally aid in the delivery of bioactive compounds to the dermis. Furthermore, spicules open pores and prevent future occlusion and consequent formation of comedones.

Spongilla spicules can be used as a resurfacing modality that rejuvenates the skin, removes old, debilitated or dead cells from the skin's outer layer without harming the younger, living cells and result in softer, smoother skin.

### EXAMPLES

The following examples provide formulations using exact amounts in grams and milliliters of each ingredient. However, these exact weights and volumes should not be considered limitations. All of the liquids used herein are aqueous based and contain low percentages of solute. Therefore, the relative weight of each volume of liquid ingredient will be considered equal to the weight of water (1 g/mL). The appended claims will therefore be expressed as ratios. For example, a composition made in accordance with the teachings of the presently invention may contain 1.5 g of substantially pure Spongilla powder, 0.5 mL hydrogen peroxide, 2 mL of 5% boric acid, 1 gram of green sea weed powder and 10 mL of floral water. This composition would then be claimed as follows: 1.5 parts of substantially pure Spongilla powder, 0.5 parts 3% hydrogen peroxide, 2 parts of 5% boric acid, 1 part of green sea weed powder and 10 parts of floral water; etc.

### Example 1

### Use of Spongilla Composition as a Dermabrasive

A sufficient amount of substantially pure spongilla powder for one application of the treatment for the face (approximately 0.5 gram of the spongilla powder for the face), chest, neck, shoulders or other area of the skin that intended to be treated was mixed with a carrier in ratios from 1:2 or greater until resulted mixture (resurfacing treatment) appears as a thin paste of fairly homogeneous consistency. Exemplary carriers include, but are not limited to, gels, water, oils, hydrogen peroxide or other liquids.

One quarter of the amount of the paste (mask) was applied to the mid-forehead by massaging in circular motions. The mask was gently spread with the fingertips in small circular movements up to the hairline using even pressure. As the mask at application area begins to dry, the steps were repeated moving down the temples, cheeks, nose and chin. A desirable mild to moderate erythema due to the increased blood circulation will develop. A tingling sensation of spicules under the skin is commonly experienced during application and after removal of the mask when treated area is touched. The mask should be massaged into the skin before it dries to optimize penetration of the spicules into the skin. The treatment may take 5-15 minutes for the face depending on the carrier used and desirable depth of resurfacing to be achieved. The neck and chest may take less time and are usually more sensitive than face. After massaging, the composition should be left to dry on the skin.

TABLE 2
Recommended Treatment Times:

| | |
|---|---|
| Face | 10-30 minutes |
| Neck | 5-20 minutes |
| Chest | 5-20 minutes |
| Scalp | 30 minutes |

For deeper resurfacing, the treated area is massaged again after the composition has dried on the skin with carrier that was used for mixing the purified spongilla powder.

After the treatment is complete, the dried dermabrasive composition is rinsed from the skin with cold or warm water. Additionally, an emollient cleanser can be used.

For the best results, heavy moisturizers should be avoided for at least 12 hours. On the 5^{th} day, the subject should wash their face using a facial scrub to remove peeling epidermis.

Use of a sunscreen with SPF 30 is necessary when exposed to the sun for at least 2 weeks after treatment.

### Example 2

### Clinical Experience

Immediately post application a healthy appearing glow was evident due to the enhanced blood circulation in the treated skin. Spicules penetrated through epidermis communicate their presence and effectiveness via a unique tingling sensation to the touch. The skin erythema and tingling sensation of the skin gradually fades over the following 12-24 hours. This unusual feedback recedes during the first two days as the spicules start shedding off. Over the next 2-4 days these microscopic spicules slowly separate old cells from underlying new dermal cells thereby increasing stratum corneum sloughing action. The skin heals and regenerates beneath the protective, but soon to be shed, outer layer of stratum corneum. In this fashion new dermal cells have both the time and an optimal milieu in which to mature prior to exposure to the harsh environment. On the fifth day these ready to shed cells were exfoliated with a common scrub. Facial resurfacing of a subject with the dermabrasive composition of the present invention is depicted in FIG. 3 (FIG. 3A: before; FIG. 3B: after).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A dermabrasive composition for skin comprising:
a substantially pure powder of a fresh water sponge of the genus Spongilla and at least one pharmaceutically acceptable excipient, wherein said composition comprises insoluble material of said substantially pure powder of Spongilla and wherein said insoluble material comprises skeletal spicules.

2. The dermabrasive composition according to claim 1 wherein said freshwater sponge is of the species Spongilla lacustris.

3. The dermabrasive composition according to claim 1 wherein said pharmaceutically acceptable excipient is a carrier gel or liquid.

4. The dermabrasive composition according to claim 1 wherein said substantially pure powder does not contain any particles larger than 0.2 mm.

5. The dermabrasive composition according to claim 1 comprising from 0.1% to 100% substantially pure Spongilla powder.

6. The dermabrasive composition according to claim 1 wherein said pharmaceutically acceptable excipient is selected from the group consisting of water, glycerin, gels, oils, waxes, emollients, cleansers, fragrances, antiseptics, anesthetics, seaweed powder, coral powder, hydrogen peroxide, enzyme gel, jojoba oil and boric acid.

7. The dermabrasive composition according to claim 6 wherein said water is selected from the group consisting of water for injection, irrigation water, distilled water, deionized water, chamomile water and calendula water.
